SEQUENCE LISTING (14 sequences)

SEQ ID NO: 1 - protein, 624 aa, Aspergillus sp.
MGRRKIEIKAIKDDRNRSVTFLKRKGGLFKKAHELAVLCSVDVAVIIFGHNKKLYEFSSCDMREALTRYQYFGPPHEHKGPEDFNGKRDDDDDEDETTPAPEDVHPTPPNPPMIPAHLPGHPGFQHVTHAPSASPPIGNGMAFAPRHGTPQPQGVSRPSSRNHLRRVSSNMGPQQHHHATPPPPPPPPQNGFTYIPNPSVYNPNAAHAMAQPPRPPQFTHYGPPGPHHQPPPPPQHHPAMPPHSMPPQSMPPHTMAPQAMPPHHQAPPHMPHPPHALSQQPPPMAMTQPPHAAIPQVAQAFLPEQGRNSMPPTFTTEQQPPPPRTVSLPEGTAPTDAMPGPMKTEGTPSPPHQRSLSSKSRSIFTPIDDRGSVLARHFGVGPPTEAGENAQTKAEGATLGPNEGKPAGLTAKPPPPPPRAATEAPRPQPVVDLKPPVRTNSGQFPPKRPQLKVQIPSENSDRGSATADSSSRDSAGNKTLTPAKANPDTGHSSVVLPPPSPSAGAILSAGAQGPPNPFARPPPPGAAPQNNSAYNNNSNIDTPISALPSRFVSDALLPSPSSFFPEWGFGRSGPDSNMLPSPLTFPTPAVQSGPGFGREDEQDKKRKSPDSGPNAEGVVKKPKT

SEQ ID NO: 2 - DNA, 4228 bp, Aspergillus sp.
tgttggaaagagggggaataatatgtaaatgagagggaaaagaggggtgggtaagcgtgaaatagtattattgtggaagaggatagagagaaagaatgaaaaaagtccgacccagaggcttcacaaaaagtaagcatgaaatgtagaggtgagactgaaggaaagcatgctacccttccattttatatctactcaacactacctcacttaagtggtatctatagtagctaaaggtgatggaactgccgggacaatcaatctattaatagggaggcggtagtcaacagccgccgttggcgagttggttagccgccgggacgatgggccaaacctcgaacgggcggcagctcttcccaccgtcggtctatctttagaataatcttctgcatcacccgcgtgtcgccgctccagccatcaatcatcctttccctttctcatccccattcattccatcatcatttcgccccccccatcccagaaattaattgactttttgttccatcacgtcgcctatctctcgtctctcgttgctcaaggtgggtttcctaaggcctcgtctttctctctcttccctttgtttccctgccctgtccatctctctctctaccgcccaccccctttgtcgtttcttcctgccgttcggatcacgcccgcgcgcgcccttctggtgtcctgttcatcccctcctccaaaataccgtcactcttcccaatctcacccctccttcctgttgctcttcccacccaacaactctctcgcatcaccaaccccctcccacacaccccccggtcgttcactatcttccctggtttttgcatattatcgccttaccttctctccgtagcggagaaagacagtacttctctccatgtggggactctagggaaatactaaatataaattctgcccacagtcttggaggttcctgccatccttttttattatttttttacaccctctaatcgcgtcccggaaatccttgcgggcggcgcttcctcgagtttcagctcgcgccgccatctcatccggacgaaccattcccccagaagctctgcttgacctgttcgcctgcgtgtttcatcgacgctcttttctgtgggattcgttcatccgctgtccctcatgggtcgcagaaagatcgagatcaaggcgatcaaggatgatcgcaaccgctcagtgtaagtgctaaagctggtggatctcttcacctctccccattaaaccctagttcgtcaagctaacgctctggtgcgcgactaggacctttctgaaacggaagggtggtctgttcaagaaggcacatgagcttgcggtcctgtgttcggttgatgtcgctgttattatctttggtcacaacaagaagctctacgaattctcgtcatgtgatatgcgagaggccctcactcgatatcaatacgtgcgttgtccacctaacccgctcgtctggaaagactgtcgttgatctgacattgccgctttcctatgcttttagtttggtcccccacatgaacacaaaggccctgaagactttaacggcaaacgcgacgatgatgatgacgaagatgagacgacgcctgcccccgaagatgtgcatcctacgcctccaaatcctcccatgattcccgcccacctgccaggtcatcccggcttccagcacgtcactcatgcgccgtctgcttccccgcccatagggaatgggatggcatttgctccgcgtcacggtacccctcaaccgcagggagtttctcggccttcgtctcggaatcatcttcgtcgggtgagctccaatatgggacctcaacagcatcaccatgcgacccctccgccgcctccgccgccgccgcaaaatggcttcacgtacattcctaatccttcggtatataaccccaacgctgctcacgccatggctcaacctccccgcccacctcagttcactcattacggaccccccgggccgcatcaccaaccccctccgcctccacagcatcatccagcgatgccgccacactccatgcccccacagtctatgcctccacatacaatggcaccgcaagccatgccgccgcaccaccaggctcctccacacatgccacacccgcctcatgcactttcacagcaaccgcctccaatggccatgacccagccgccccatgccgccatcccgcaggtcgcgcaagcgtttctcccagagcaaggacgaaattccatgcctccgacgtttacaacagaacagcaaccaccgccgccacgaacagtgtcgcttcctgaaggaaccgctccgacagacgcaatgccaggtcccatgaagacggaaggaaccccatccccgccgcaccagcgatctctctcttccaagtctcgcagtatcttcacacccattgacgaccgaggctccgtattggcgcgccattttggcgtaggtcctccgacggaggcgggagagaatgcgcaaacgaaggctgaaggagcaacactcggaccaaacgaagggaagcctgctgggctaactgcaaagccccctcctccacctccccgagcggccacggaagccccgcgtccccagccggtagttgacctgaagccgcctgtgcgtacaaacagcggccagttcccgcctaagcgcccgcagttgaaggtacagataccgagtgagaattcggaccggggcagcgcaacggcagattcatcgtcacgtgattctgcggggaacaaaacgctcacgcctgccaaagcgaatccagacaccggtcactcgagcgtggtattgcccccgccctcgccgtctgcgggtgcgatattaagtgcaggggctcaagggccgccaaatccttttgcccggccaccgcctccgggagcagcaccacagaacaacagtgcatacaacaataatagcaatatagatacgccaatctctgctcttcctagtcggttcgtctcggatgcgcttctcccgtcaccgtccagtttctttccggaatggggctttgggcggtctggaccagacagcaacatgttgcccagtcccttgacgttccctacgccggcggtacaaagtggccccgggtttgggcgcgaggatgagcaagacaaaaagcgcaaaagccctgatagtggacccaatgcggaaggggtagtgaagaaaccaaagacgtaatccactccatattgttacaactactagactgatgattgtggtattttgttgctggggtgatcaagtcccccgtgtttcatatatggcatctcaaggcgttggcgaaagctacttttggtttccttaattgggatgggaaagttcgaatgaggctcgttttgtttcccctggatcctttttttgtcctccggttctgaaccagtaccaagtccttccttgttttgtgataagggtttccgatttaactttataccccttccttttattgttattgccatggtcgtttttggcagttgacctagctatccatcgatttctctccaaagtccgaaaccaaatgagaatggtgcgataaatctttccttttcctacctatttcctttttctcttgctgttttgctttttttcgctggggactttgaagatgatagaagacacataatattattatattaagtgtttcaatcttctagtcaagggtggctttctctttcatatattactatgttcttattattttggaataaaagtaaaaaaaaaaaaggtgtacataacaaagatctgcgatgtgcaactaggagaattaattcaatagtaggtttcttagtttgaatactgtggttcaccgcacgtcccgtgttccggtgcttctattataagaagagtagatagtagggatttaaattgattgatagtccgttgccgtgaacctaaacgttaactctaaatttcagttttgtcgcgcgcacgagtcatgacatcaactgactttctaccttgtttatctgatagatagagttggatgatggttatgcaagggtggggttttggttcacaacacaagttactgtcgtcatataagtataataaaacagtaactatcgaatgatatgatattatagtcaatttcattgtggtaaacatttgatttatgtatgcagatcatagtatcatatggtatgatgctactaagcacgagtgccattttctaattactactagtaaagtaagtatctcaaccagtctgtctgtcaggtagtattattagattttaagtatttagatacatcatca

SEQ ID NO: 3 - DNA, 1875 bp, Aspergillus sp.
atgggtcgcagaaagatcgagatcaaggcgatcaaggatgatcgcaaccgctcagtgacctttctgaaacggaagggtggtctgttcaagaaggcacatgagcttgcggtcctgtgttcggttgatgtcgctgttattatctttggtcacaacaagaagctctacgaattctcgtcatgtgatatgcgagaggccctcactcgatatcaatactttggtcccccacatgaacacaaaggccctgaagactttaacggcaaacgcgacgatgatgatgacgaagatgagacgacgcctgcccccgaagatgtgcatcctacgcctccaaatcctcccatgattcccgcccacctgccaggtcatcccggcttccagcacgtcactcatgcgccgtctgcttccccgcccatagggaatgggatggcatttgctccgcgtcacggtacccctcaaccgcagggagtttctcggccttcgtctcggaatcatcttcgtcgggtgagctccaatatgggacctcaacagcatcaccatgcgacccctccgccgcctccgccgccgccgcaaaatggcttcacgtacattcctaatccttcggtatataaccccaacgctgctcacgccatggctcaacctccccgcccacctcagttcactcattacggaccccccgggccgcatcaccaaccccctccgcctccacagcatcatccagcgatgccgccacactccatgcccccacagtctatgcctccacatacaatggcaccgcaagccatgccgccgcaccaccaggctcctccacacatgccacacccgcctcatgcactttcacagcaaccgcctccaatggccatgacccagccgccccatgccgccatcccgcaggtcgcgcaagcgtttctcccagagcaaggacgaaattccatgcctccgacgtttacaacagaacagcaaccaccgccgccacgaacagtgtcgcttcctgaaggaaccgctccgacagacgcaatgccaggtcccatgaagacggaaggaaccccatccccgccgcaccagcgatctctctcttccaagtctcgcagtatcttcacacccattgacgaccgaggctccgtattggcgcgccattttggcgtaggtcctccgacggaggcgggagagaatgcgcaaacgaaggctgaaggagcaacactcggaccaaacgaagggaagcctgctgggctaactgcaaagccccctcctccacctccccgagcggccacggaagccccgcgtccccagccggtagttgacctgaagccgcctgtgcgtacaaacagcggccagttcccgcctaagcgcccgcagttgaaggtacagataccgagtgagaattcggaccggggcagcgcaacggcagattcatcgtcacgtgattctgcggggaacaaaacgctcacgcctgccaaagcgaatccagacaccggtcactcgagcgtggtattgcccccgccctcgccgtctgcgggtgcgatattaagtgcaggggctcaagggccgccaaatccttttgcccggccaccgcctccgggagcagcaccacagaacaacagtgcatacaacaataatagcaatatagatacgccaatctctgctcttcctagtcggttcgtctcggatgcgcttctcccgtcaccgtccagtttctttccggaatggggctttgggcggtctggaccagacagcaacatgttgcccagtcccttgacgttccctacgccggcggtacaaagtggccccgggtttgggcgcgaggatgagcaagacaaaaagcgcaaaagccctgatagtggacccaatgcggaaggggtagtgaagaaaccaaagacgtaa

SEQ ID NO: 4 - DNA, 36 bp, Artificial Sequence
  Description of Artificial Sequence Synthetic oligonucleotide
ataagaatgcggccgcaaagtccgacccagaggctt

SEQ ID NO: 5 - DNA, 28 bp, Artificial Sequence
  Description of Artificial Sequence Synthetic oligonucleotide
tgctctagagggacagcggatgaacgaa

SEQ ID NO: 6 - DNA, 27 bp, Artificial Sequence
  Description of Artificial Sequence Synthetic oligonucleotide
tgctctagaagtggacccaatgcggaa

SEQ ID NO: 7 - DNA, 28 bp, Artificial Sequence
  Description of Artificial Sequence Synthetic oligonucleotide
ggggtacccccacccttgcataaccatc

SEQ ID NO: 8 - DNA, 21 bp, Artificial Sequence
  Description of Artificial Sequence Synthetic oligonucleotide
ctacctgacagacagactggt

SEQ ID NO: 9 - DNA, 19 bp, Artificial Sequence
  Description of Artificial Sequence Synthetic oligonucleotide
tgcccagtcccttgacgtt

SEQ ID NO: 10 - DNA, 19 bp, Artificial Sequence
  Description of Artificial Sequence Synthetic oligonucleotide
aatgtcaattccagcagcg

SEQ ID NO: 11 - DNA, 19 bp, Artificial Sequence
  Description of Artificial Sequence Synthetic oligonucleotide
ttagcgctctgagtgtcgg

SEQ ID NO: 12 - DNA, 20 bp, Artificial Sequence
  Description of Artificial Sequence Synthetic oligonucleotide
cgatgcgatccaggaagatg

SEQ ID NO: 13 - DNA, 30 bp, Artificial Sequence
  Description of Artificial Sequence Synthetic oligonucleotide
atgacgtttcccaccgagagtagagaatga

SEQ ID NO: 14 - DNA, 31 bp, Artificial Sequence
  Description of Artificial Sequence Synthetic oligonucleotide
ctctcggtgggaaacgtcatatcaggatagc

(19) United States
(12) Patent Application Publication
(10) Pub. No.: US 2009/0047701 A1
(43) Pub. Date: Feb. 19, 2009

(54) METHOD FOR SCREENING ANTIFUNGAL AGENTS

(75) Inventors: Arthur Franciscus Johannes Ram, Hillegom (NL); Robbert Antonius Damveld, Leiden (NL); Mark Arentshorst, Noordwijk (NL); Patricia Ann van Kuyk, Wageningen (NL); Cornelis Antonius Maria Jacobus Johannes van den Hondel, Gouda (NL)

Correspondence Address: FOLEY AND LARDNER LLP;SUITE 500, 3000 K STREET NW, WASHINGTON, DC, 20007, US

(73) Assignee: STICHTING VOOR DE TECHNISCHE

(21) Appl. No.: 11/813,189

(22) PCT Filed: Dec. 23, 2005
(86) PCT No.: PCT/NL05/50094

§ 371 Date: Nov. 12, 2007

(30) Foreign Application Priority Data
Dec. 30, 2004 (EP) 04078572.7

(51) Int. Cl. C12Q 1/02; C12N 1/14; C12N 1/15; C07H 21/00; C07K 14/00

(52) U.S. Cl. 435/29; 435/254.1; 435/254.11; 435/256.1; 435/256.5; 536/23.74; 530/350

(57) ABSTRACT

The present invention relates to a fungus which produces substantially no functional protein with regulating activity on the transcription of genes which are involved in cell wall stress response. It also relates to a method for screening for antifungal agents, in particular to antifungal agents which target the cell wall. It also relates to a kit for screening for antifungal agents which disturb cell wall biogenesis.

### FIELD OF THE INVENTION

The present invention relates to a method for identification of antifungal agents and their mode of actions. In particular, it relates to cell wall disturbing antifungal agents.

### BACKGROUND OF THE INVENTION

The cell wall of fungi is an essential component of the fungal cell. By interfering with the synthesis or assembly of the fungal cell, the cell will lyse and die and therefore the cell wall is an ideal antifungal target. The fungal cell wall contains several classes of macromolecules, including β1,3-glucan, β1,6-glucan, chitin, cell wall mannoproteins and in some cases α1,3 or α1,3-α1,4-glucan. Both the presence of these components and the crosslinking of the several components to each other to form a rigid cell wall are essential. Thus antifungals that interfere with the synthesis of one of these components or antifungals that interfere with the crosslinking of those compounds are interesting as antifungal agents. Antifungals are grouped into five groups on the basis of their site of action: (1) azoles, which inhibit the synthesis of ergosterol (the main fungal sterol); (2) polyenes, which bind to fungal membrane sterol, resulting in the formation of aqueous pores through which essential cytoplasmic materials leak out; (3) allylamines, which block ergosterol biosynthesis, leading to accumulation of squalene (which is toxic to the cells); (4) flucytosine, which inhibits protein synthesis and (5) candins (inhibitors of the fungal cell wall), which function by inhibiting the synthesis of beta 1,3-glucan (the major structural polymer of the cell wall) (Balkis et al., 2002, Drugs 62 (7): 1025-1040). Only this latter class of candins are antifungal that specifically inhibit cell wall biosynthesis.

Although the class of candins are an interesting and potential valuable antifungal drug there is clearly a need for additional drugs, because laboratory experiments using S. cerevisiae have shown that mutants resistant to candins can spontaneously arise. Despite the recent entrance of glucan synthase inhibitors in clinical trials, knowledge of mechanisms of resistance against candins in patients is lacking. Furthermore, candins display a poor antifungal activity towards some fungi eg. C. neoformans and its activity towards non-Aspergillus molds have not been established today. Finally, tolerance against candins have been reported through activation of the PKC1 signalling cascade which offers the fungal cell a pathway to become resistant to candins. Therefore is it clear that there is a need for additional antifungals.

An antifungal agent that interferes with fungal cell wall biosynthesis and acts at the outside of the cell is highly preferable, because fungal cells possess several mechanisms to remove antifungal agents from the cell, e.g. by exporting them via plasma membrane localized transporters, which also decrease the efficiency by which a antifungal can act. Currently, new antifungal screens are based on in vitro assays to screen antifungal compounds to affect biosynthesis of the cell wall. WO2004/048604 claims a method for the identification of compounds that affect GPI-anchor biosynthesis, CA2218446 claims a method for the identification of antifungal which inhibits beta1,6-glucan In addition, method are disclosed in the article, to identify antifungals in vitro (e.g. Cercosporamide (Sussman et al., Eukaryotic Cell 3(4): 932-943). These in vitro screens are relatively difficult to perform, are likely to identify only antifungal compound that act inside the cell and therefore have to cross the membrane, and molecules that inhibit a reaction in vitro, may not have that effect in vivo, which indicates negative aspects of in vitro screening.

The use of reporter strains to screen for antifungal compounds in vivo have been claimed in WO03020922 and WO2004/057033. These reporter based screening methods require sophisticated fluorescent microscopes and handling which limit the high throughput possibilities of the methods at the moment. There is clearly a need for alternative screening methods which are simpler and more cost-effective.

### SHORT DESCRIPTION OF THE FIGURES

FIG. 1

Sequence alignment and phylogenetic relationship of MADS-box containing transcription factors. A. Alignment of the 102 amino acids fragment containing the MADS-box and MEF2/SAM domain of MADS-box transcription factors. Amino acid residues that are identical in all protein sequences are shown in a black background. Residues printed in a light-, intermediate- or dark-grey background are resp ≧33%, ≧50% or ≧75% identical. Proteins aligned are: Aspergillus nidulans (AN2984.2; EAA63555 and AN8676.2; EAA60098), Fusarium graminearum (FG09339.1; EAA76082 and FG8696.1; EAA70796), Neurospora crassa (NCU02558.1; EAA36453 and NCU07430.1; EAA35381), Ustilago maydis (UM05323.1; EAK86572 and UM01124.1; EAK81831), A. niger (RlmA; (AY704272), Magnaporthe grisea (MG02773.4; EAA47530), A. fumigatus (AfRlmA; a_fumigatus|chr_{—}0|TIGR.5237|59 ATG at 1893428), Saccharomyces cerevisiae (ScSmp1p; CAA85143, ScRlm1p; AAB68210, ScMcm1p; CAA88409, ScArg80p; CAA88408), Homo sapiens (HsSRF; AAH48211, HsMef 2A; AAH13437), Mus musculus (MmMef2D; AAH11070) and Arabidopsis thaliana (AtAgamous; NP_{—}567569). Proteins were aligned using the multiple sequence alignment tool in DNAMAN version 4.0. An optimal alignment was performed with the following settings: a gap open penalty of 40, and a gap default penalty of 10, other parameters were default. Indicated with black lines are: the 57 amino acids MADS-box region, required for DNA binding, the 28 amino acids MEF2 domain and the 24 amino acids SAM domain. B. Homology tree of the 102 amino acids fragment containing the MADS-box and MEF2/SAM domain of MADS-box transcription factors. The tree was created with DNAMAN using the alignment as obtained as described above using the output tree optional to visualize sequence identities.

FIG. 2

Disruption of the rlmA gene in A. niger. A. Schematic representation of: the rlmA wildtype locus (top); the plasmid, pΔRlmA, used for disruption (middle), and the deleted locus of the ΔrlmA strain (bottom). Abbreviations: B, BglI; K, KpnI; N, NdeI; No, NotI; X; XbaI; 1, probe. B. Southern blot analysis of the wild-type (WT) and rlmA deletion strains (#1 and #5). Genomic DNA was digested with NdeI. Digestion of WT DNA is predicted to result in a 7.0 kb fragment, whereas digestion of genomic DNA from a deletion strain should result in a 3.6 kb fragment. The blot was probed with an approximately 300 bp XbaI-NcoI TrlmA fragment as indicated in the figure.

FIG. 3

Cell wall integrity response in the wild-type (N402) and the ΔrlmA strain. Northern analysis of the agsA, and 18S messenger levels. Strains were grown in shake flask until small germ tubes were formed. Subsequently CFW (+) or an equal volume of water (−) was added to the cultures. RNA was extracted at the timepoints indicated above the Northerns, t=time in minutes. The probes used; for agsA a ˜0.6 kb EcoRI agsA fragment was isolated from pGEMT-agsA (Damveld et al, unpublished vector); the 18S ribosomal probe was isolated as a 2 kb BglI fragment from pMN1 (Borsuk et al., 1982 Gene 17, 147-152.)

FIG. 4

Sensitivity of the ΔrlmA strain towards different compounds. Growth curves of wild-type (N402) and ΔrlmA strains. Spores were grown in Complete medium for 24 hrs at 37° C. in the presence of various concentrations of antifungals. Data are represented as the mean and standard error of the mean obtained from four replicates. The dotted line with open squares represents the growth curve of the rlmA deletion strain and the solid line with closed circles represent the parental (N402) strain.

### DETAILED DESCRIPTION

The present invention relates to a fungus which produces substantially no functional protein with a sequence according to SEQ ID No.1 or homologues thereof.

One of the advantages of a fungus according to the invention is that it can be used for the identification of antifungal agents which disturb cell wall biogenesis. In contrast to state of the art methods, the use of the fungus allows for the design of a simple identification method which does not require expensive tools.

Another advantage is that it can be expected that antifungal agents identified using the method of the invention will give good results in toxicity tests, since they act on the cell wall, a cell component which is not present in human cells. Therefore, they are very likely toxic to the fungus, but not to its host. In addition, an antifungal compound that interferes with the synthesis or assembly of the cell wall is highly preferable, since the antifungal compound does not have to be transported across the plasmamembrane. This transport might be a bottleneck for the antifungal activity.

As used herein, the term “functional” means that there is regulating activity towards the transcription of downstream target genes activated in response to cell wall stress. Cell wall stress can be induced by several forms, eg. the addition of cell wall related antifungal compounds e.g treatment with glucanases, Calcofluor White, Caspofungin or Congo red, by physical treatment e.g. heat shock or mechanical stress or by the use of cell wall mutants. Target genes in Aspergillus niger include, agsA and gfaA which encode the α-1,3-glucan synthase protein and the glutamine-fructose-6-phosphate amidotransferase respectively.

A fungus which produces “substantially no functional protein” produces not enough protein to have regulatory activity on the transcription of downstream target genes involved in cell wall stress response.

In one embodiment, the fungus produces no protein with a sequence according to SEQ ID No.1, or homologue thereof, at all as indicated by mRNA levels determined in Northern blot analysis. In another embodiment, the fungus produces less than 10%, preferably less than 5, 4, 3, 2, or 1% of the protein level produced by a parent strain as indicated by mRNA levels determined in Northern blot analysis.

In this context, a “parent strain” is a wild type strain or a wild type-like strain which is capable of making a protein with regulating activity on the transcription of down stream target genes activated in response to cell wall stress and which produces normal levels of the protein. The skilled person will understand that the “normal level” will be dependent on environmental or culture conditions. The fungus which produces substantially no functional protein may be called a mutant of the parent strain.

As used herein, the term “fungus” refers to filamentous fungi and yeast, with the provison that the yeast does not belong to the species Saccharomyces cerevisiae. Species which are included are species belonging to the genus of Aspergillus, Fusarium, Penicillium, Schizosaccharomyces, Candida, Neurospora, Magnaphtha, Ustilago, Graminarium, Cryptococcus, Histoplasma, Exophiala, and Crysosporium. In a preferred embodiment, the fungus is a filamentous fungus. For instance, a fungus belonging to the genus of Aspergillus or Chrysosporium, in particular a fungus of the species Aspergillus niger or Chrysosporium lucknowense.

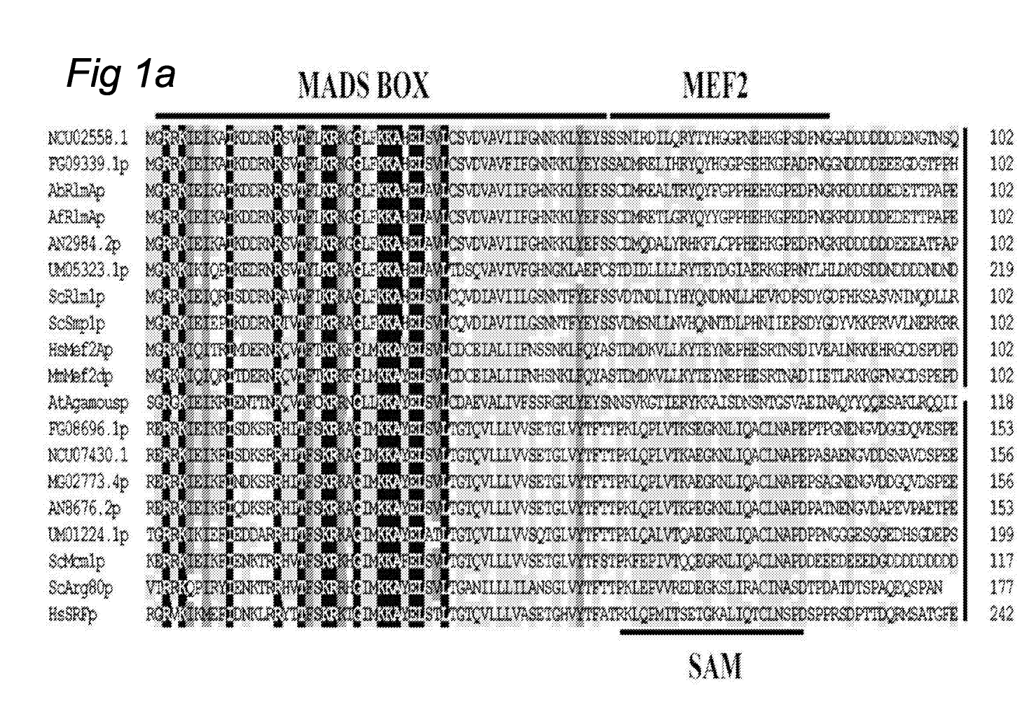

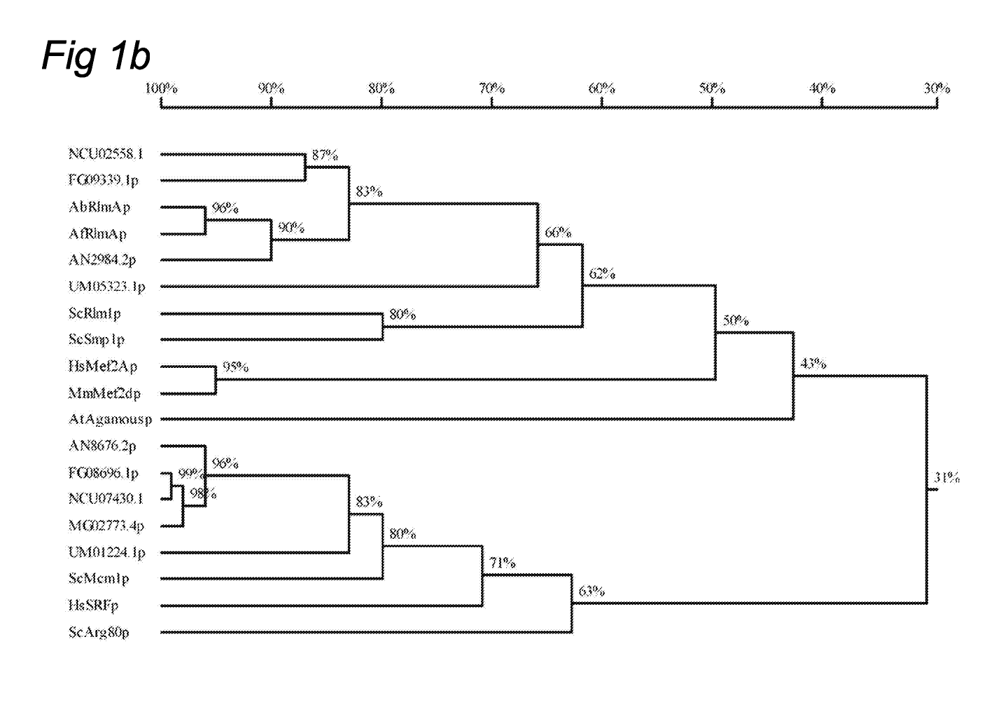

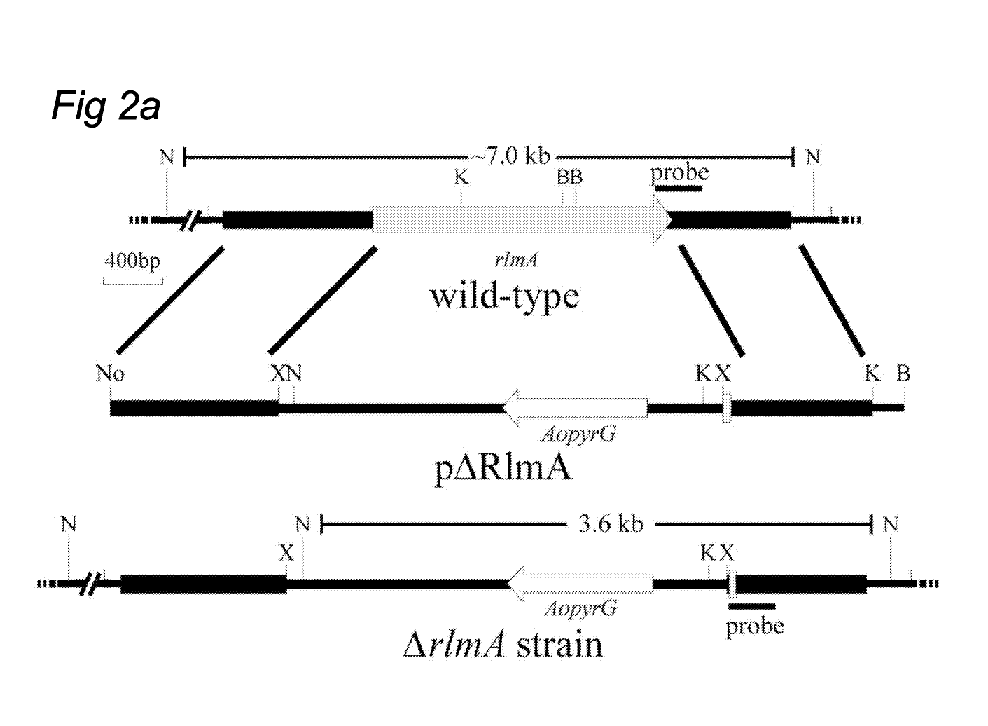

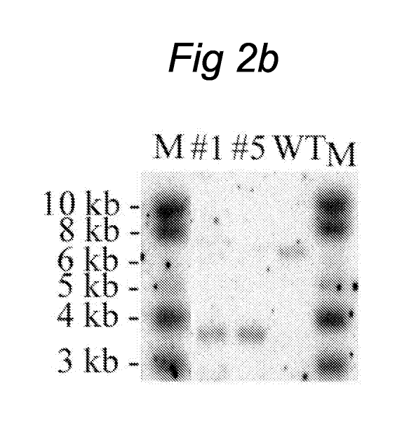

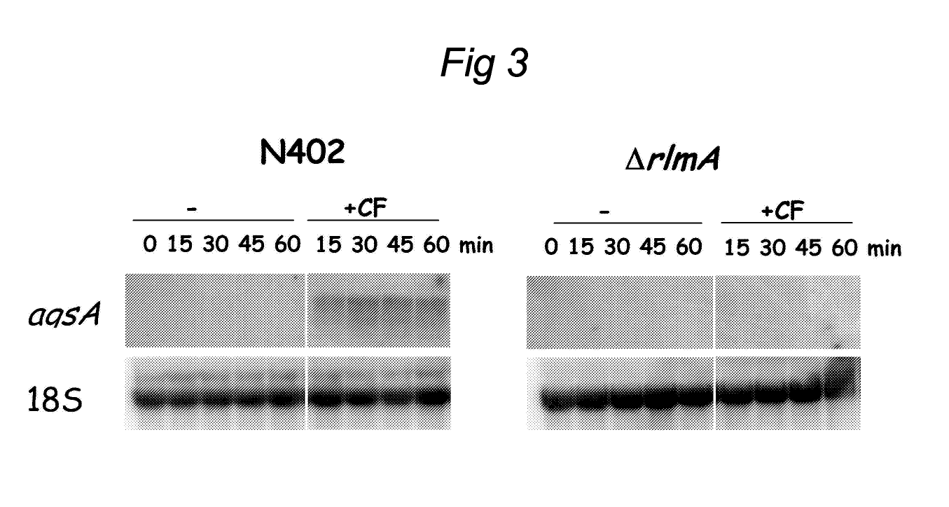

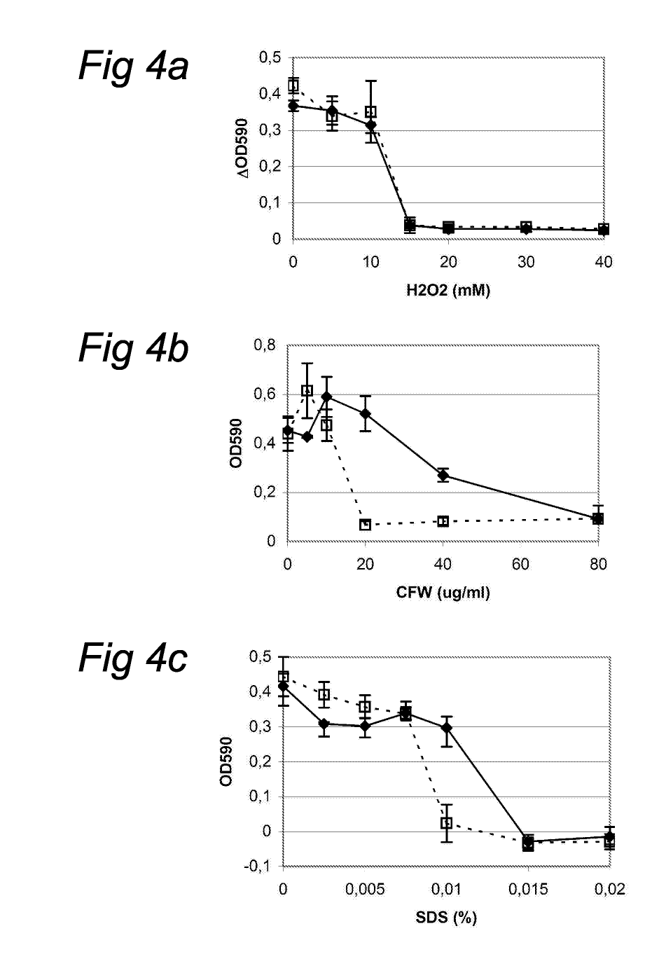

The protein represented by SEQ ID NO.1 belongs to the family of MADS-box transcription factor proteins (Dodou and Treisman 1997, Mol. Cell. Biol. 17 (4), p 1848-1859 and Huang et al, 2000, EMBO J. 19 (11), pp 2615-2628). See also FIG. 1A. In the context of this invention, homologues of the protein which is represented by SEQ ID NO:1 have a sequence which is homologous to SEQ ID No 1. A homologous sequence is encoded by a polynucleotide which also contain a MADS-box domain and which ends up in the homology tree of FIG. 1B if the DNAMAN alignment program as described in this application is used. Suitable polynucleotides are depicted in SEQ ID NO. 2 and 3. The protein represented by SEQ ID NO.1 and homologues thereof, provided that Scrlm1p of S. cerevisiae is not included, are collectively called proteins of the invention. Proteins of the invention have regulating activity on the transcription of downstream target genes involved in cell wall stress and are characterised by a MADS-box domain with a conserved MADS-box motif RX₁KX₅IX₅RX₂TX₂KRX₂GX₂KKAX₁ELX₂L, (wherein in Xₙ X denotes any amino acid and n the number of amino acids). In one embodiment of the invention, proteins of the invention contain a MEF2 domain in addition to the above mentioned MADS-box motif. In another embodiment of the invention, proteins of the invention, contain a SAM domain in addition to the above mentioned MADS-motif. In a preferred embodiment of the invention, proteins of the invention are represented by SEQ ID NO.1 or are homologues thereof which contain a MEF2 domain. A representative example of a protein of the invention is rlmA of Aspergillus niger.

State of the arts methods may be used for decreasing the amount of functional protein with a sequence according to SEQ ID No.1 in the fungus. These methods include methods which interfere with replication, transcription, translational or which interfere at post-translational level. Methods which may be used for this purpose include gene deletion and gene disruption strategies, construction of point mutations or dominant negative alleles and RNA interference. These methods may involve compounds such as anti-sense RNA, siRNA, miRNA, hnRNA, antibodies, including intrabodies, or fragments thereof; peptide and non-peptide inhibitors. Functional protein expression levels may also be affected by modification of the transcript, such as by phosphorylation, acetylation, methylation or hydroxylation.

In one embodiment, the amount of functional protein is decreased by down regulating the gene encoding the protein by deletion of one or more nucleotides in the gene. A preferred target of deletion is the MADS-box domain. Preferably, at least 30, 40, or 50 nucleotides of this domain, more preferably all nucleotides of this domain are deleted. In yet another embodiment, not just the MADS-box, but the whole gene of the protein is deleted.

Inactivation of the gene by gene deletions may be introduced by methods known in the art, and include the use of fungal transformation of gene deletion or gene disruption constructs, UV-mutagenesis; or other methods to substitute, delete or add one or more or nucleotides to the wild type locus.

A special and very effective way of deletion is gene disruption by inserting foreign DNA into the structural gene in order to disrupt transcription. This can be effected by the creation of a genetic cassette comprising the foreign DNA to be inserted, e.g. a fungal marker gene, flanked by sequences which have a high degree of homology to a portion of the gene to be disrupted. Introduction of the cassette into the host cell will result in insertion of the fungal marker gene into the structural gene by homologous recombination and thus in disruption of the structural gene.

In another aspect of the invention, a fungus according to the invention is used in a method for the identification of or screening for antifungal agents which disturb cell wall biogenesis. The terms “screening for” and “identification of” are used interchangeably in this context. These types of antifungal agents are applicable in many fields in industry, especially in the feed and food industry, the chemical industry or in the pharmaceutical industry.

In one embodiment, the fungus is used in a method for the identification of antifungal agents which disturb cell wall biogenesis comprising:
contacting a potential antifungal agent with a fungus according to the invention; and
measuring the growth of the fungus for an appropriate time at an appropriate temperature,
   whereby a decreased growth rate in comparison to a parent fungus, e.g. no growth at all, is indicative of the positive identification of an antifungal agent.

The potential antifungal agent may be contained in a solid or liquid medium on or in which the fungus can grow. It may be added before or after germination. It may be added in any suitable formulation form, e.g. as a powder or as spray. Suitable examples of growth media include Complete Medium consisting of Aspergillus Minimal Medium (MM) (Bennett and Lasure, 1991) with the addition of 10 g l⁻¹ yeast extract and 5 g l⁻¹ casamino acids. Liquid medium might be solidified by the addition of 0.2-2% agar. The potential antifungal agent may be dissolved in water, DMSO or ethanol and can be added most easily to liquid medium.

In one embodiment of the method of the invention, the growth rate of the fungus is monitored every 1-2 hours by determining the optical density of a particular microtiterplate well containing fungal spores and an antifungal for at least 20 hours, preferably at least 30, 35 or 40 hours. In a more preferred embodiment, the growth is monitored for at least 2 or 3 days. The appropriate temperature depends on the fungus, but is typically between about 25 and 37° C. In a preferred embodiment, the temperature is in the range of 30 to 37 degrees C.

Another aspect of the invention is a kit containing a fungus according to the invention. The kit may also contain in a separate container a parent fungus which is capable of making a protein with regulating activity on the transcription of down stream target genes involved in cell wall stress response and, optionally, an inducer of cell wall stress as a positive control. Compounds which are suitable to be used as inducers of cell wall stress include Calcofluor white, SDS, tunicamycine, caspofungin and, a moderate one, benomyl. Such kit may also contain a negative control (non-inducer), such as hydrogenperoxide.

### EXAMPLES

### Materials and Methods

### Strains, Culture Conditions and Transformations

Aspergillus niger N402 (cspA1 derivative of ATCC9029; Bos et al., 1988, Current Genetics 14, 437-443) and the pyrG negative derivative of N402, AB4.1 (van Hartingsveldt et al., 1987, Mol. Gen. Genet. 206(1), 71-75) were used throughout this study. Aspergillus strains were grown in Aspergillus Minimal Medium (MM) (Bennett and Lasure, 1991, More Gene Manipulations in Fungi Academic Press, San Diego. pp. 441-447) or Aspergillus Complete Medium (CM) consisting of minimal medium with the addition of 10 g l⁻¹ yeast extract and 5 g l⁻¹ casamino acids. Growth medium was supplemented with 10 mM uridine (Serva) when required. Transformation of A. niger was as described by Punt and van den Hondel (Punt and van den Hondel (1992) Methods in Enzymology, pp. 447-457) using lysing enzymes (L1412, Sigma) for protoplast formation. Conidiospores were obtained by harvesting spores from a CM-plate after 4-6 days of growth at 30° C., using 0.9% NaCl. The bacterial strain used for transformation and amplification of recombinant DNA was Escherichia coli XL1-Blue (Stratagene, La Jolla, Calif.). XL1-Blue was transformed using the heat shock protocol as described by Inoue et al., (1990) Gene 96, 23-28.

### Molecular Biological Techniques

Chromosomal DNA of A. niger was isolated as described by Kolar (Kolar et al., 1988, Gene 62, 127-134). Alternatively, chromosomal DNA was isolated using the FastPrep FP120 (Bio101). First, A. niger spores were grown in Fast-prep tubes containing 1 ml CM and 0.3 gram acid washed glass beads. After growth for 16 hours at 37° C. the mycelium was spun down, medium was removed, 500 μl cold extraction solution (2:2:1 mixed, TNS; 40 mM tri-isonaphtalene sulphonic acid, PAS; 0.70 M P-aminosalycillic acid, and RNB; 1.0 M Tris-HCl pH 8.5, 1.25 M NaCl, 0.25 M EDTA) and 500 μl phenol:cholorform:isoamyl alcohol (25:24:1 v/v %) was added. Vials were vigorously shaken in a FastPrep FP120 (Bio101) twice for 30 seconds at speed 6.0 and cooled for five minutes on ice between runs. Both Southern and Northern blot analyses were carried out as described by Sambrook et al (Sambrook et al., 1989, Molecular Cloning: a Laboratory Manual, 2nd ed. Cold Spring Harbor Laboratory Press, Plainview N.Y.). [α-³²P]dCTP-labelled probes were synthesised using Rediprime II DNA labelling System (Amersham Pharmacia Biotech) according the instructions of the manufacturer. RNA was extracted from mycelium snap-frozen in liquid nitrogen using TRIzol reagent (InVitrogen). Total RNA (10 μg) was incubated with 3.3 μl 6 M glyoxal, 10 μl DMSO and 2 μl 0.1 M sodium phosphate buffer, pH 7.0, in a total volume of 20 μl for one hour at 50° C. to denature the RNA. RNA electrophoresis was performed in a SEA-2000 (Elchrom Scientific) at 10° C. PCR was performed on a PTC-100 Programmable Thermal Controller (MJ Research, Inc) using Super Taq (HT Biotechnology LTD) or when required Expand High Fidelity PCR system (Roche). Primers were obtained from Isogen and are listed in Table 1. For ligation the Rapid DNA Ligation Kit (Boehringer Mannheim) was used. Sequencing was carried out with a Perkin Elmer ABI PRISM 310 sequencer using the ABI prism Big Dye Terminator Cycle Sequencing Ready Reaction Kit (Applied Biosystems). Restriction enzymes were obtained from InVitrogen and used according to the protocol supplied by the manufacturer.

### Cell Wall Stress-Inducing Conditions and Cell Wall Analysis

A. niger spores were inoculated in 50 or 100 ml CM at a spore density of 1×10⁷ spores ml⁻¹ and grown for 5 hours at 37° C. and 300 rpm. After the spores had germinated, germlings were treated with a cell wall-stress inducing compound (200 μg ml⁻¹ Calcofluor White, CFW) by adding the compound from a freshly prepared stock solution (20 mg ml⁻¹ CFW) or an equal volume of water was added as a control. At specific time points after the addition of CFW, germlings were harvested rapidly using a sieve with a 20 μm aperture (Endecotts) and frozen with liquid nitrogen prior to the isolation of RNA or cell walls.

Sensitivity towards various compounds was assayed in 96-well microtiter plates (Nunc, art. 164588) using a Perkin Elmer HTS-7000 Bioassay reader. A series of concentrations of stress-inducing compounds (CFW, Caspofungin, Hydrogen-peroxide, SDS) were prepared in 100 μl milliq in a 96-well plate, and 100 μl spore solution (˜2×10⁴ spores) in 2× complete medium was added to 100 μl stress-inducing solution. The microtiter plates were incubated at 37° C. and the OD₅₉₀ was measured every 2 hours.

Construction of the rlmA::pyrG Deletion Plasmid

The DNA sequence encoding the A. niger RlmA transcription factor was obtained from DSM (DDBJ/EMBL/GenBank databases accession number: AY704272 rlmA) and was used to generate a disruption construct by PCR. The complete rlmA encoding gene, including 1122 bp of the promoter sequence and 1074 bp of the terminator sequence is shown as SEQ ID No.1. rlmA contains an open reading frame (ORF) of 4228 bp which is interrupted by two introns of 82 and 75 bp and encodes a 624 amino acid protein. The 5′ promoter region of rlmA was amplified using primers RlmAP1 and RlmAP2 (Table 1). The 3′ terminator region was amplified using RlmAP3 and RlmAP4. PCR products of 1020 and 903 bp were obtained, digested with NotI and XbaI or XbaI and KpnI, respectively, and used in a three way ligation using pBluescript-KS which had been digested with NotI and KpnI to give pRLM1. The rlmA deletion construct was made by inserting a 2.7 kb XbaI-XbaI fragment from pAO4-13 (de Ruiter-Jacobs et al., 1989, Current Genetics 16, 159-163), containing the pyrG gene from A. oryzae into the unique XbaI site of pRLM1 to give pΔRlmA. The disruption cassette was linearized with NotI/BglI and transformed to A. niger pyrG strain AB4.1. RlmAP7 and pAO-9 were used to identify putative gene deletion mutants by PCR on genomic DNA isolated from fungi grown in 2 ml fast-prep tubes (Bio101, Cat # 5076-400). The primer sequence of RlmAp7 (Table 1) is localized outside the 3′ gene disruption construct and primer pAO-9 (Table 1) anneals on the A. oryzae pyrG gene. A double cross-over and thus deletion of the rlmA locus would result in the amplification of a 1.1 kb fragment. Genomic DNA of 11 pools each containing 20 transformants were analysed by PCR. All pools gave a 1.1 kb PCR product, indicating that they all contained at least one disruption strain. Because pool 1 produced the most PCR product, genomic DNA from individual transformants of this pool was further analysed, by repeating the PCR reaction using RlmAp7 and pAO-9, and also by PCR with primer pair RlmAP7 and RlmAP8. The primer RlmAP8 is located within rlmA and should give a PCR product of ˜1100 bp if the rlmA gene is still intact. In this pool five out of the 20 transformants showed product with the gene deletion primer set (RlmAP7-pAO-9) and no product with the rlmA primer set (RlmAP7-RlmAP8) indicating that those five transformants contain a deletion of the rlmA gene. Southern analysis was used to confirm deletion of the rlmA gene.

TABLE 1
Primers used.

| | |
|---|---|
| Primer Name | Sequence (5′ to 3′) |

| | |
|---|---|
| RlmAp1 | ATAAGAATGCGGCCGCAAAGTCCGACCCAGAGGCTT |
| RlmAP2 | TGCTCTAGAGGGACAGCGGATGAACGAA |
| RlmAP3 | TGCTCTAGAAGTGGACCCAATGCGGAA |
| RlmAP4 | GGGGTACCCCCACCCTTGCATAACCATC |
| RlmAP7 | CTACCTGACAGACAGACTGGT |
| RlmAPS | TGCCCAGTCCCTTGACGTT |
| pAO-9 | AATGTCAATTCCAGCAGCG |

Restriction sites are underlined

### Example 1

### The A. niger rlmA Gene is Required for the Induced Expression of agsA in Response to Calcofluor White (CFW) Induced Cell Wall Stress

To examine the role of RlmA during the induction of agsA the rlmA gene was disrupted. A disruption cassette containing the pyrG gene from A. oryzae flanked by ˜1-kb promoter and 1-kb terminator region of rlmA was constructed as described in Materials and methods and is shown in FIG. 2. After transformation of the linearized construct, putative rlmA deletion strains were first identified by PCR. Correct deletion of the rlmA gene in PCR positive transformants by a double cross-over event, was confirmed with Southern blot analysis.

To determine the role of A. niger RlmA in the activation of agsA in response to cell wall stress, the expression of agsA was analysed in both the wild-type strain (N402) and the rlmA deletion strain (ΔrlmA) after CFW treatment. N402 was grown for five hours and the ΔrlmA strain was grown for 5.5 hours until both strains had formed a small germtube. Both strains were treated with 200 μg/ml CFW. RNA was isolated after 0, 15, 30, 45 and 60 minutes from cultures treated with and without CFW. Results are shown in FIG. 3.

The expression level of the agsA gene was already induced 15 minutes after CFW addition, indicating a rapid transcriptional response to the presence of CFW. Since no agsA mRNA could be detected in the ΔrlmA strain after treatment with CFW, the induction of agsA seems dependent on the RlmAp transcription factor. This result provides further evidence for an important role of a Rlm1p dependent signal transduction cascade in A. niger which mediates the cell wall remodelling response.

### Example 2

### Hypersensitivity of the ΔrlmA Strain Towards Cell Wall Related Antifungal Compounds

The sensitivity of the wild-type and the ΔrlmA strain towards various compounds was also measured by determining fungal growth in a microtiter plate based growth assay. The rlmA deletion strain displayed a hypersensitive phenotype towards several cell wall disturbing compounds, such as CFW and SDS. Sensitivity of the ΔrlmA strain was slightly, but reproducible enhanced towards the cell wall biosynthesis disturbing compounds, Caspofungin and tunicamycin, and towards the microtubule inhibitor benomyl, which is likely to affect cell wall biosynthesis indirectly by influencing the transport of cell wall components to the cell surface. Importantly, the ΔrlmA strain displayed no hypersensitive phenotype towards the negative control H₂O₂ (FIG. 4). This shows that a higher sensitivity of the rlmA deletion strain to antifungal compounds in comparison to the sensitivity of the wild type strain to the same antifungal is indicative of a cell wall related mode of action of the particular antifungal of antifungal extract.

1-19. (canceled)

20. An isolated fungus substantially free of protein capable of regulating transcription of one more genes involved in cell wall stress response.

21. The fungus according to claim 20 comprising a gene for the protein, wherein the gene contains a deletion rendering the protein incapable of regulating transcription of one more genes involved in cell wall stress response.

22. The fungus according to claim 20, wherein a gene for the protein is deleted in whole or in part.

23. The fungus according to claim 20, wherein the fungus is a mutant of a parent fungus, the parent fungus comprising a protein capable of regulating transcription of one or more genes involved in cell wall stress response.

24. The fungus according to claim 20 wherein the fungus is a filamentous fungus.

25. The fungus according to claim 21, wherein the fungus is a filamentous fungus.

26. The fungus according to claim 24, wherein the filamentous fungus is of genus Aspergillus, Fusarium, Ustilago, Magnaporthe or Chrysosporium.

27. The fungus according to claim 26, wherein the filamentous fungus is of species Aspergillus niger, Fusarium graminearum, Ustilago maydis, Magnaporthe grisea or Chrysosporium lucknowense.

28. The fungus according to claim 20, wherein the protein comprises a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids.

29. The fungus according to claim 20, wherein the protein comprises a MEF2 or a SAM domain.

30. The fungus according to claim 20, wherein the protein is represented by SEQ ID No.1.

31. The fungus according to claim 28, wherein the protein further comprises a MEF2 or a SAM domain.

32. A polynucleotide sequence comprising a polynucleotide which:
(a) encodes a protein capable of regulating transcription of one more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids;
(b) encodes a protein of SEQ ID NO. 1, or a homologue thereof, capable of regulating transcription of one more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids and; or
(c) is represented by SEQ ID NO. 2.

33. An isolated protein capable of regulating transcription of one or more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids.

34. An isolated protein comprising SEQ ID NO. 1, or a homologue thereof, wherein the protein is capable of regulating transcription of one or more genes involved in cell wall stress response.

35. A method for the identification of antifungal agents comprising:
(a) contacting an agent with a fungus substantially free of protein capable of regulating transcription of one more genes involved in cell wall stress response;
(b) contacting a potential antifungal agent with a wild-type fungus comprising protein capable of regulating transcription of one more genes involved in cell wall stress response; and
(c) measuring growth of the fungus from (a) and the fungus from (b) for at least 20 hours,
(d) identifying as antifungal agents those agents which retard the growth rate of the fungus from (a) relative to the fungus from (b).

36. The method according to claim 35, wherein the growth is measured up to 3 days.

37. The method according to claims 35, wherein the fungus is contacted with the potential antifungal agent provided in or on a solid or liquid medium.

38. A kit for screening for antifungal agents that disturb cell wall biogenesis, the kit comprising:
(a) a fungus substantially free of protein capable of regulating transcription of one more genes involved in cell wall stress response;
(b) an isolated protein capable of regulating transcription of one or more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids;
(c) an isolated protein comprising SEQ ID NO. 1, or a homologue thereof, wherein the protein is capable of regulating transcription of one or more genes involved in cell wall stress response;
(d) a nucleotide encoding a protein capable of regulating transcription of one more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids;
(e) a nucleotide encoding a protein of SEQ ID NO. 1, or a homologue thereof, capable of regulating transcription of one more genes involved in cell wall stress response, the protein comprising a MADS-box domain with a MADS-box motif RX₁KX₅IX₅RX₂TX₂KRX₂GX₂KKAX₁ELX₂L, wherein X denotes any amino acid and the subscripted numeral thereafter denotes the number of X amino acids;
(f) a nucleotide represented by SEQ ID NO. 2; or
(g) a combination thereof.

39. A kit according to claim 38 further comprising a parent fungus capable of making a protein with regulating activity on the transcription of downstream target genes.

40. A kit according to claim 38 further comprising an inducer of cell wall stress response.

41. A kit according to claim 40 wherein the inducer of cell wall stress response is Calcofluor white, SDS or benomyl.
